(19) United States
(12) Plant Patent Application Publication
(10) Pub. No.: US 2011/0191927 P1
(43) Pub. Date: Aug. 4, 2011

(54) Hippeastrum plant named 'OPAL STAR'

(50) ;

(75) Inventors: Dorit Sandler-Ziv, Nes Ziona (IL); Avner Cohen, Nes Ziona (IL); Aurel Ion, Rehovot (IL); Corneliu Fintea, Azur (IL); Marina Baizerman, Bat Yam (IL); Ahuva Daous, Mazkeret Batia (IL)

(73) Assignee: STATE OF ISRAEL, MINISTRY OF AGRICULTURE & RURAL DEVELOPMENT, AGRICULTURAL RESEARCH ORGANIZATION

(21) Appl. No.: 12/657,941

(22) Filed: Jan. 29, 2010

(51) Int. Cl. A01H 5/00

(52) U.S. Cl. PLT/402

(57) ABSTRACT

A new and distinct Hippeastrum interspecific hybrid plant named ‘OPAL STAR’ particularly characterized by star-shaped flower, measuring about 16 cm to 18 cm in length and about 12 to 16 cm in diameter, and with a unique, color pattern of red (RHS 42A) with yellow-greenish (RHS 146D) stripes and dark red (RHS 46A) veining.

### LATIN NAME OF THE GENUS AND SPECIES OF THE PLANT CLAIMED

Hippeastrum Herb.

### VARIETY DENOMINATION

‘OPAL STAR’

### BACKGROUND OF THE INVENTION

The present invention relates to a new and distinct interspecific hybrid variety of Hippeastrum, botanically known as Hippeastrum Herb., syn. Amaryllis, of the Amaryllidaceae family, and hereinafter referred to by the cultivar name ‘OPAL STAR’.

The Hippeastrum genus is native to tropical and subtropical regions of South America and the Caribbean, and includes more than 80 different species and several hundred hybrids.

The new Hippeastrum ‘OPAL STAR’ is a product of a controlled breeding program conducted by the inventors, Dorit Sandler-Ziv, Corneliu Fintea, Aurel Ion, Avner Cohen, Marina Baizerman and Ahuva Daus, in Bet Dagan, Israel. The objective of the breeding program was to develop a new Hippeastrum cultivar with excellent ornamental characteristics, adapted to bulb production under Israeli climatic conditions and suitable for both cut flower and pot production worldwide.

The new Hippeastrum ‘OPAL STAR’ originated from a cross made by the inventors in 2000 in Bet Dagan, Israel. The female or seed parent is the unpatented, wild-type Hippeastrum papillio designated WT30. The male or pollen parent is the unpatented, interspecific Hippeastrum hybrid designated 97/4 (H. vittatum ‘WHITE’×wild-type H. cybister designated WT40). The new Hippeastrum ‘OPAL STAR’ was selected by the inventors as a single flowering plant from the progeny of the stated cross in a controlled environment in 2003 in Bet Dagan, Israel.

Asexual propagation of the new Hippeastrum ‘OPAL STAR’ using the “twin-scaling” method (division of the mother bulb into about 12 vertical segments which are subsequently subdivided into 5 to 6 twin-scale units was first performed in September, 2004, in Bet Dagan, Israel. The twin-scale units are dipped for 15 minutes in 0.2% Benlet and 0.5% Marpan to prevent fungal infection, then the segments are air dried for 3 to 4 hours and placed in moist Vermiculite no. 2 (8% to 10% moisture) and incubated at 22° C. to 24° C. for 3 to 4 months. The resulting bulblets are planted in coco-peat and Tuff 0.8 (80:20%) and grown for 2 years until they flower for the first time. Of the twin scales first propagated in September, 2004, the resulting bulbs first flowered in the winter of 2007, and demonstrated that the combination of characteristics as herein disclosed for the new cultivar are firmly fixed and retained through successive generations of asexual propagation. The new cultivar reproduces true to type.

### BRIEF DESCRIPTION OF THE INVENTION

The following traits have been repeatedly observed and are determined to be unique characteristics of ‘OPAL STAR’, which in combination distinguish this Hippeastrum as a new and distinct cultivar:
1. star-shaped flower, measuring about 16 cm to 18 cm in length and about 12 to 16 cm in diameter, and with a unique, color pattern of red (RHS 42A) with yellow-greenish (RHS 146D) stripes and dark red (RHS 46A) veining.

Plants of the new Hippeastrum ‘OPAL STAR’ differ from plants of the female or seed parent, the unpatented, wild-type Hippeastrum papillio designated WT30 , in the following characteristics described in Table 1.

TABLE 1

| | | Female Parent |
|---|---|---|
| | New Cultivar | H. papillio WT30 |
| Characteristic | ‘PAL STAR’ | (unpatented) |
| Plant Height | 60 cm to 80 cm | 18 cm to 24 cm |
| Number of flowers per | 4 to 5 | 2 to 3 |
| inflorescence | | |
| Outer tepal shape | Elliptic | Oblanceolate |
| Outer tepal, Upper side | Red | Whitish |
| Main color | | |
| Outer tepal, Upper side | Dark red | Maroon red |
| Secondary vein | | |

Of the many commercial cultivars known to the present inventor, the most similar to the new Hippeastrum ‘OPAL STAR’ is the Hippeastrum hybrid (leopoldii-type) ‘Red Lion’ (patented, grant number unknown, and differs from the new Hippeastrum ‘OPAL STAR’ in the following characteristics described in Table 2:

TABLE 2

| | | Comparison Cultivar |
|---|---|---|
| | New Cultivar | ‘Red Lion’ |
| Characteristic | ‘OPAL STAR’ | (patented) |
| Plant height | 60 cm to 80 cm | 38 cm to 60 cm |
| Number of stems per | 1 to 3 | 2 to 3 |
| bulb per year | | |
| Scape length | 55 cm to 70 cm | 38 cm to 50 cm |
| Number of flowers per | 4 to 5 | 2 to 4 |
| inflorescence | | |
| Flower Shape | Star shape | Rounded |
| Flower Throat | Covered with | Covered with |
| | numerous white and | numerous white and |
| | red hairs | yellowish hairs |
| Outer tepal | Red, RHS 42A | Red, RHS 43A |
| Upper side, Main color | | |
| Outer tepal | Dark red with thin light- | Glossy red with short |
| Under side, Main color | yellow stripes | greenish veins |
| Filament | | |
| Color of upper part | Yellow | Red |
| Color of lower part | Yellow-green | Red |
| Anther color | Greenish | Light yellow |
| Fruit/Seed | None | Present |

### BRIEF DESCRIPTION OF THE PHOTOGRAPHS

The accompanying photographs illustrate the overall appearance of the new Hippeastrum cultivar ‘OPAL STAR’ showing the colors as true as is reasonably possible with colored reproductions of this type. Colors in the photographs may differ slightly from the color values cited in the detailed botanical description, which accurately describe the color of ‘OPAL STAR’.

FIG. 1 shows a close-up side view perspective of the typical fully open flowers produced by ‘OPAL STAR’.

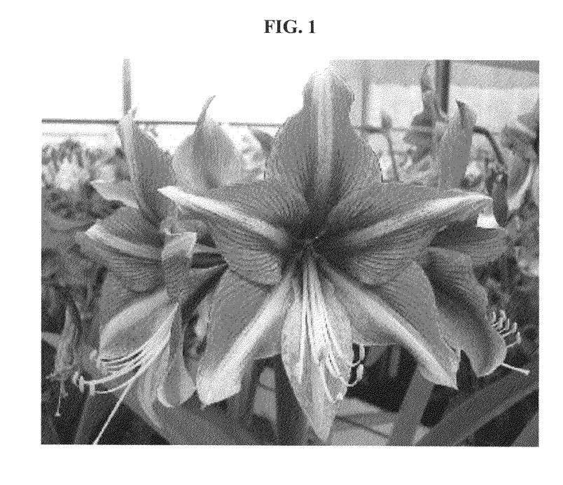

FIG. 2 shows a close-up side view comparison of (1) the typical fully open flowers produced by ‘OPAL STAR’ (top) to (2) the typical fully open flowers produced by the female parent, the unpatented, wild-type Hippeastrum papillio designated WT30.

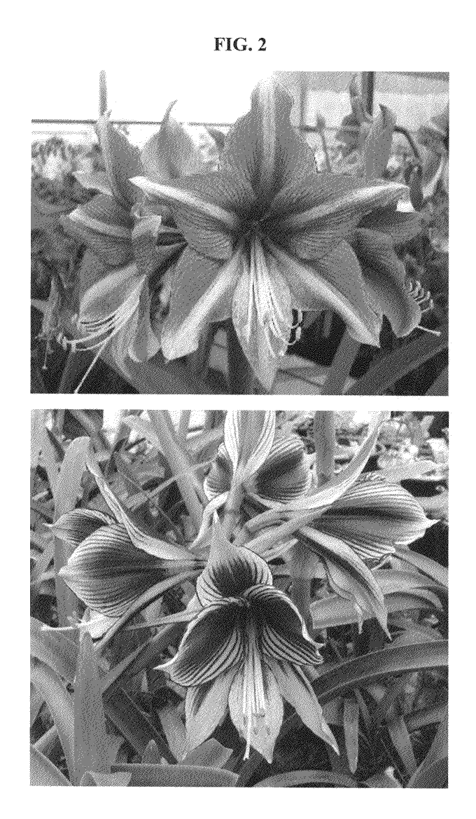

FIG. 3 shows a close-up side view comparison of (1) the typical fully open flowers produced by ‘OPAL STAR’ (top) to (2) the typical fully open flowers produced by the male or pollen parent, the unpatented interspecific Hippeastrum hybrid designated 97/4 (H. vittatum ‘WHITE’×wild-type H. cybister designated WT40).

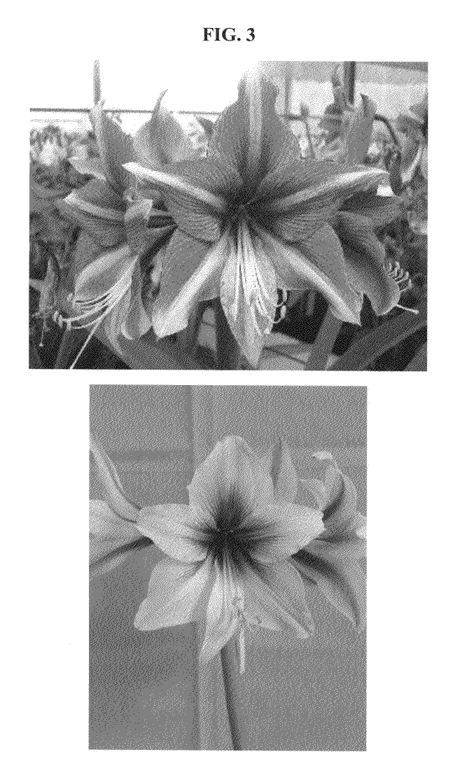

FIG. 4 shows a close-up side view comparison of (1) the typical fully open flowers produced by ‘OPAL STAR’ (top) to (2) the typical fully open flowers produced by the unpatented, wild-type Hippeastrum cybister (WT40) (one of the cultivars crossed to produce the male or pollen parent, the unpatented interspecific Hippeastrum hybrid designated 97/4.)

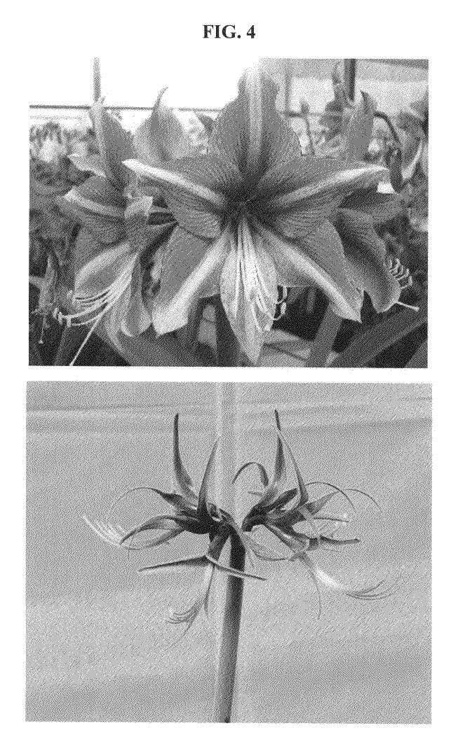

FIG. 5 shows a close-up side view comparison of (1) the typical fully open flowers produced by ‘OPAL STAR’ (top) to (2) the typical fully open flowers produced by the commercial cultivar, Hippeastrum hybrid (leopoldii-type) ‘Red Lion’.

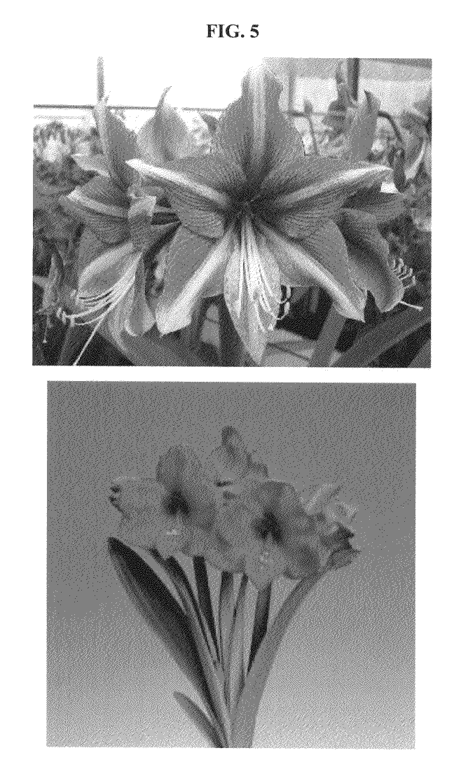

### DETAILED BOTANICAL DESCRIPTION

The new Hippeastrum cultivar ‘OPAL STAR’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe plants of ‘OPAL STAR’ as grown in a greenhouse in Bet Dagan, Israel, under conditions which closely approximate those generally used in commercial practice.

Mother bulbs of ‘OPAL STAR’ (about 28 cm to 30 cm in circumference) are cut into 12 vertical segments which are subsequently subdivided into 5 to 6 twin-scale units. The twin-scale units are dipped for 15 minutes in 0.2% Benlet and 0.5% Marpan to prevent fungal infection, then the twin-scale units are air dried for 3-4 hours and placed in moist Vermiculite no. 2 (8% to 10% moisture) and incubated at 22° C. to 24° C. for 3 to 4 months. The resulting bulblets are planted and grown in raised, heated soil beds (soil temperature maintained at an average temperature of about 20° C. to 22° C.) in coco-peat and Tuff 0.8 (80:20%) and grown for 2 years until they flower for the first time.

Plants of ‘OPAL STAR’ have been grown in an insect-proof greenhouse, covered with a permanent polycarbonate roof with movable polyethylene curtains, and under 50% shading. Drip irrigation is administered at 80-100 m3/ha and a liquid fertilize is applied of (NPK 5:3:8) 50 ppm. Plants of ‘OPAL STAR’ are grown with (1) an average winder air temperature of about 12° C. to 25° C. during the day and about 6° C. to 12° C. during the evening and (2) an average summer air temperature of about 22° C. to 38° C. during the day and about 18° C. to 22° C. during the evening. Plants of ‘OPAL STAR’ reach full growth about 6 to 10 weeks after planting in the summer season of cured bulbs.

Color references are made to The Royal Horticultural Society Colour Chart (R.H.S.), 1986 edition, except where general colors of ordinary significance are used. Color values were taken under daylight conditions at approximately noon in full sunlight in Bet Dagan, Israel. The age of the ‘OPAL STAR’ plants described is about 6 to 10 weeks after planting of a cured bulb in the summer season.
Classification:
Botanical: Hippeastrum Herb.
Parentage:
Female or seed parent: Unpatented, wild-type Hippeastrum papillio designated WT30.
Male or pollen parent: Unpatented, interspecific Hippeastrum hybrid designated 97/4. (H. vittatum ‘WHITE’×wild-type H. cybister designated WT40).
Propagation:
Type: “Twin-scaling” method (division of the mother bulb into vertical segments and twin-scale units).
Roots:
   Habit and description.—Fleshy; inserted around basal plate of bulb.
   Length.—Up to 20 cm.
   Diameter.—About 2 mm to 4 mm.
   Color.—Off-white, RHS 158C.
Plant:
   Appearance.—Perennial, herbaceous; 1-3 scapes developing sequentially with a complement of 5 to 8 leaves.
   Growth habit.—Erect.
   Habit.—Leaves are slightly arching and scapes vertical.
   Vigor.—Strong, fast.
   Crop time.—Following asexual propagation, 2 years of growing time are required to produce a flowering plant. Plants of ‘RUBY STAR’ reach full growth about 6 to 10 weeks after planting of cured bulbs in late autumn.
   Size at maturity.—Height: About 60 cm to 80 cm. Spread: About 50 cm to 60 cm.
Bulb (described after harvesting and curing, before planting):
   Structure.—Tunicated bulb composed entirely of enlarged leaf bases.
   Overall shape.—Spherical.
   Shape in cross section.—Rounded.
   Shape in longitudinal section.—Elliptical to oblate.
   Length.—About 7.5 cm to 9.5 cm.
   Diameter.—About 9.5 cm to 10.2 cm.
   Texture.—Irregular.
   Color.—Of flase: Yellow-white, closest to RHS 158C Of dry scale: Greyed-orange, RHS 165B.
   Harvest time of bulbs.—Beginning of August in Bet Dagan, Israel. Lifted bulbs are dipped for 15-20 minutes in 0.2% Benlet and 0.5% Marpan solution and then air dried for 10 days.
   Storage time.—Stored in 9° C. during the second half of August until first half of November (10-12 weeks) in Bet Dagan, Israel.
Foliage:
   Arrangement and attachment.—Basal from scale; single.
   Quantity per plant.—Typically 5 to 8.
   Overall shape of leaf.—Linear.
   Apex shape.—Acute.
   Base shape.—Parallel.
   Shape of cross-section.—V-shape.
   Margin.—Entire.
   Margin undulation.—None.
   Texture.—Upper surface: Smooth; medium glossiness Under surface: Glabrous; very strong glossiness.
   Pubescence.—None.
   Mature leaf length.—About 65 cm to 85 cm.
   Mature leaf width.—About 65 mm to 80 mm.
   Mature leaf color.—Upper Surface: Dark green, RHS 137A; Under Surface: Dark green, RHS 137C.
   Venation.—Pattern: Parallel, inconspicuous venation.
Inflorescence description:
Appearance: One umbel per scape with 4 to 5 single, star-shaped flowers that open sequentially and are symmetrically arranged in a horizontal plant around center of umbel.
Natural blooming period: In spring in Bet Dagan, Israel.
Flowering response time: After planting cured bulb, plants typically flower within about 6 to 10 weeks.
Flowering longevity: On the plant: about 7 to 10 days, depending on temperature; As cut flower: about 5 to 8 days.
Inflorescence fragrance: None.
Inflorescence type: Umbel.
Umbel size:
   Height.—About 20 cm to 25 cm.
   Diameter.—About 15 cm to 20 cm.
Quantity of flower buds and flowers per umbel: About 4 to 5 .
Flower bud:
   Shape.—Broad, elliptic.
   Length.—About 9 cm to 11 cm.
   Width.—About 2.5 cm to 3 cm.
   Texture.—Smooth.
   Color.—Ground color, medium yellow-green, RHS 146D, with greyed-purple veins, RHS 183A.
Scape:
   Appearance.—Erect, leafless, hollow.
   Aspect.—Erect.
   Strength.—Strong.
   Twisting.—Slightly twisted towards flower.
   Shape in cross-section.—Elliptical.
   Length.—About 55 cm to 70 cm.
   Diameter.—Upper Part: About 23 mm to 26 mm Middle Part: About 26 mm to 32 mm Basal Part: About 30 mm to 32 mm.
   Texture.—Glabrous.
   Glaucosity.—Strong.
   Color.—Dark green, RHS 137C.
   Intensity of anthocyanin at base.—Medium.
Flower:
   Description.—An outer whorl of 3 tepals and an inner whorl of 3 tepals.
   Rate of opening.—Flowers fully opened about 2 to 3 days after tepal separation; all flowers open per umbel in about 8 to 14 days.
   Attitude.—Outward.
   Shape.—Star-shaped.
   Size.—Length: About 16 cm to 18 cm. Diameter: About 12 cm to 16 cm.
   Throat.—Covered with numerous white and red hairs.
Tepals:
   Number and arrangement.—Six tepals that are trimerous, slightly overlapping and arranged in 2 whorls. An outer whorl of 3 tepals and an inner whorl of 3 tepals.
   Outer tepals.—Overall shape: Elliptic. Apex: Obtuse with slight twisting of tip. Base: Obtuse. Margin: Entire with medium strong undulation. Length: About 10 cm to 11 cm Width: About 50 mm to 52 mm Texture: Upper surface: smooth; Under surface: smooth. Color (Mature): Upper surface: Main color: red, RHS 42A; Middle zone: Light yellow-green, RHS 146D; and Secondary vein, dark red, RHS 46A Under surface: Main color, dark red, RHS 46A, with thin yellow-green stripes, RHS 145A; and Middle zone, dark red, RHS 46A, with thin yellow-green stripes, RHS 145A.
   Inner tepals.—Description: One central lower tepal and two lateral tepals Central Tepal: number: 1 Overall shape: Spatulate. Apex: Acute with no twisting of tip. Base: Acute Margin: Entire with weak undulation. Length: About 9.5 cm to 10 cm Width: About 32 mm to 35 mm Texture: Upper surface: smooth; Under surface: smooth. Color (Mature): Upper surface: Main color: Red, RHS 46C; Middle zone: Light yellow-green, RHS 145B; and Secondary vein: Red, RHS 46C Under surface: Main color: Light yellow-green RHS 145D; Middle zone: Greyed-purple, RHS 183C; and Secondary vein: Red, RHS 46 C. Lateral tepal: Number: 2 Overall shape: Ovate. Apex: Obtuse with slight twisting of tip. Base: Obtuse Margin: Entire with strong undulation. Length: About 9 cm to 11 cm Width: About 40 mm to 45 mm Texture: Upper surface: smooth; Under surface: smooth. Color (Mature): Upper surface: Main color: red, RHS 42A; Middle zone: light yellow-green, RHS 146D; and Secondary vein: dark red, RHS 46A Under surface: Main color, dark red, RHS 46A, with thin yellow-green stripes, RHS 145C; Middle zone: dark red, RHS 46A, with thin yellow-green stripes, RHS 145C; and Secondary vein: none.
Pedicel:
   Strength.—Strong.
   Attitude of flower.—Semi upright.
   Length.—About 50 mm to 80 mm.
   Diameter.—About 5 to 7 mm.
   Texture.—Smooth.
   Color.—Medium green, with strong anthocyanin over color.
Reproductive organs:
Androecium:
   Stamen.—Number: 6. Arrangement: Appressed to lowest tepal, distal ends upwardly curved.
   Anthers.—Color: Yellowish-green in unripe stage, RHS 145C. Shape: Elongated. Length: About 8 mm to 10 mm.
   Filaments.—Length: About 95 mm to 105 mm. Diameter: About 2 mm to 3 mm. Color: Upper part: Yellow, RHS 2D; Lower part: Yellow-green, RHS 145B.
   Pollen.—Amount: Abundant. Color: Yellow-green, RHS 150B.
Gynoecium:
   Pistils.—Quantity: 1 Length: About 10 mm to 13 mm.
   Stigmas.—Shape: Three-lobed. Color: White.
   Style.—Length: About 10 mm to 13 mm Diameter: About 2 mm Color: Upper part, Yellow, RHS 2D; Lower part: Very light yellow-green, RHS 145C.
   Ovary.—Color: Green, with medium anthocyanin coloration.
Fruit/seed: None.
Disease/pest resistance: None tested or observed.
Disease/pest susceptibility: None tested or observed.

What is claimed is:

1. A new and distinct Hippeastrum interspecific hybrid plant named ‘OPAL STAR’, as illustrated and described herein.
